(19) United States
(12) Patent Application Publication
(10) Pub. No.: US 2020/0276168 A1
(43) Pub. Date: Sep. 3, 2020

(54) Sustained Release Pharmaceutical Compositions and Methods of Use

(71) Applicant: Qrono, Inc., Pittsburgh, PA (US)

(72) Inventor: Sam Rothstein, Pittsburgh, PA (US)

(21) Appl. No.: 16/781,797

(22) Filed: Feb. 4, 2020

Related U.S. Application Data
(63) Continuation of application No. 16/162,335, filed on Oct. 16, 2018, now abandoned.
Application No. 16/162,335 is a continuation of application No. 15/349,199, filed on Nov. 11, 2016, now abandoned.
(60) Provisional application No. 62/341,384, filed on May 25, 2016.
(60) Provisional application No. 62/254,015, filed on Nov. 11, 2015.

(51) Int. Cl. A61K 31/427; A61K 45/06; A61K 9/16; A61K 9/50; A61K 9/00; A61K 9/107

(52) U.S. Cl. CPC A61K 31/427; A61K 45/06; A61K 9/1647; A61K 9/5031; A61K 9/0024; A61K 9/1075; A61K 9/5084

(57) ABSTRACT

Disclosed herein are methods and pharmaceutical compositions for sustained release of anticancer agents. Such pharmaceutical compositions may provide a burst-free, sustained release of one or more anticancer agents and may include a plurality of poly(lactic-co-glycolic) acid (PLGA) microparticles encapsulating the one or more anticancer agents.

### CROSS REFERENCE TO RELATED APPLICATIONS

This application is a continuation of abandoned U.S. application Ser. No. 16/162,335, filed on Oct. 16, 2018, which is a continuation of abandoned U.S. application Ser. No. 15/349,199, filed on Nov. 11, 2016, which claims priority to expired U.S. Provisional Application No. 62/341,384, filed on May 25, 2016 and entitled “SUSTAINED RELEASE PHARMACEUTICAL COMPOSITIONS AND METHODS OF USE” and to expired_U.S. Provisional Application No. 62/254,015, filed on Nov. 11, 2015 and entitled “SUSTAINED RELEASE PHARMACEUTICAL COMPOSITIONS AND METHODS OF USE,” all of which are incorporated herein by reference.

### BACKGROUND

Present modes of chemotherapy drug delivery, such as oral delivery, and intramuscular, intravenous, and subcutaneous injections may result in high or low serum concentrations of the drug. In addition, the drug may have a shortened half-life in the blood. In some cases, achieving therapeutic efficacy with these standard administrations requires large doses of medications that may result in toxic side effects. The technologies relating to controlled drug release have been attempted in an effort to circumvent some of the pitfalls of conventional therapy. The aims of such technologies are to deliver medications on a continuous and sustained manner. Additionally, local control drug release applications are site or organ specific. The efficacy of controlled release of a chemotherapy drug would be enhanced if the cytotoxic drug could be preferentially transported to draining lymph nodes, which are a primary site of tumor metastasis.

### SUMMARY

Disclosed herein are methods and compositions for sustained release of anticancer agents. In one embodiment, a pharmaceutical composition for burst-free, sustained release of one or more anticancer agents includes a plurality of smaller poly(lactic-co-glycolic) acid (PLGA) microparticles of 0.5-5 micrometers in diameter encapsulating a first anticancer agent, and a plurality of larger microparticles of 10-50 micrometers in diameter encapsulating a second anticancer agent. In some embodiments, the first anticancer agent and the second anticancer agent may be same. In other embodiments, the first anticancer agent and the second anticancer agent may be different.

In another embodiment, a pharmaceutical composition includes a first population of small microparticles encapsulating one or more anticancer agents having a mean particle diameter of about 0.5 to about 10 micrometers, and a second population of large microparticles encapsulating one or more anticancer agents having a mean particle diameter of greater than about 10 micrometers.

In one embodiment, a pharmaceutical composition for burst-free, sustained release of one or more anticancer agents beginning after 2 weeks of administration includes a plurality of poly(lactic-co-glycolic) acid (PLGA) microparticles encapsulating the one or more anticancer agents. In some embodiments, the composition may include microparticles of various sizes. In some embodiments, the ratio of poly-lactide to poly-glycolide in the microparticles is about 30:70 to about 99:1 by weight.

In another embodiment, a method of treating a subject with cancer includes administering a therapeutically effective amount of a pharmaceutical composition, wherein the composition provides a burst-free, sustained release of one or more anticancer agents for a duration of 5 weeks or more. The composition may include a plurality of poly(lactic-co-glycolic) acid (PLGA) microparticles encapsulating the one or more anticancer agents. In some embodiments, the composition may include microparticles of various sizes. In some embodiments, the smaller microparticles are phagocytosed by antigen presenting cells and are transported to draining lymph nodes. In some embodiments, the larger microparticles that are not phagocytosed remain at the site of injection. In some embodiments, the delayed onset of burst-free, sustained release from smaller and larger microparticles protects antigen presenting cells and other phagocytic cells from the cytotoxic effects of anticancer agents that would otherwise hinder cell migration, trafficking or motility.

In a further embodiment, a method of treating a subject with cancer includes administering a therapeutically effective amount of a pharmaceutical composition having a first population of small microparticles encapsulating one or more anticancer agents having a mean particle diameter of about 0.5 to about 10 micrometers, and a second population of large microparticles encapsulating one or more anticancer agents having a mean particle diameter of greater than about 10 micrometers.

In another embodiment, a method of treating a subject with cancer includes administering a therapeutically effective amount of a pharmaceutical composition comprising microparticles having a bimodal particle size distribution, wherein the microparticles encapsulate one or more anticancer agents.

In a further embodiment, a pharmaceutical composition includes microparticles having a polydisperse particle size distribution, and wherein the microparticles encapsulate one or more anticancer agents. In some embodiments, the microparticles may have a mean particle size diameter from about 20 micrometers to about 40 micrometers, and a standard deviation of about 20 micrometers.

In an additional embodiment, a method of treating a subject with cancer includes administering a therapeutically effective amount of a pharmaceutical composition comprising microparticles having multimodal particle size distribution, wherein the microparticles encapsulate one or more anticancer agents.

### BRIEF DESCRIPTION OF THE FIGURES

FIGS. 1A-1E show kinetics of the drug release. FIG. 1A shows that 12 kDa PLGA with a poly-lactide to poly-glycolide ration of 50:50 (L:G) microparticles release drug between days 1 and 12. FIG. IB shows that 20 kDa 75:25 microparticles release drug starting at day 21. FIG. 1C shows that 101 kDa 65:35 microparticles begin releasing drug at day 32; and FIG. 1D shows that 47 kDa 75:25 microparticles begin releasing drug at day 40. FIG. 1E shows a profile for all four of these microparticles (FIGS. 1A-1D) in a single formulation, projected to sustain drug release at a near constant rate until at least day 50. These release kinetics data reflect drug dissolution from microparticles incubated in phosphate buffered saline under gentle agitation at 37° C.

FIG. 2 shows reduction in tumor volume in a head and neck squamous cell carcinoma xenograft model in mice treated with a single dose of sustained release microparticle formulation. Over four weeks of monitoring a single intratumoral injection of sustained release microparticles according to the present invention (QR206) significantly out-performs an equivalent bolus of unformulated drug, reducing tumor volume 4-fold. Both high and low dose long acting formulations according to the present invention achieve statistically significant reductions in tumor volume relative to the unformulated drug control (EpoD) with a payload equal to a low dose sustained release formulation (T-test, * p<0.04, ** p<0.02). A vehicle only control (Control) displayed a higher tumor growth rate than the unformulated drug.

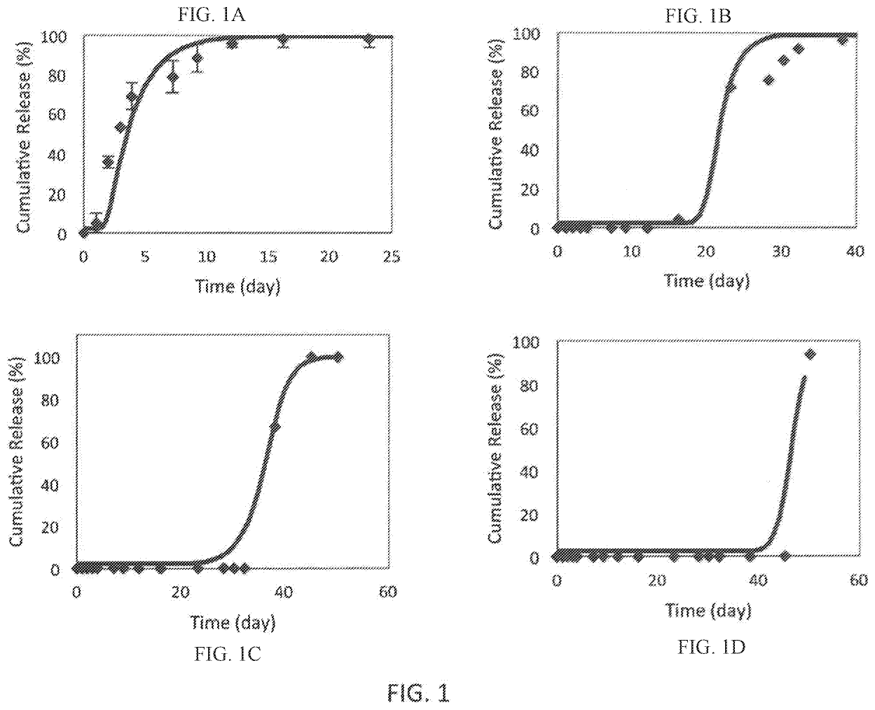

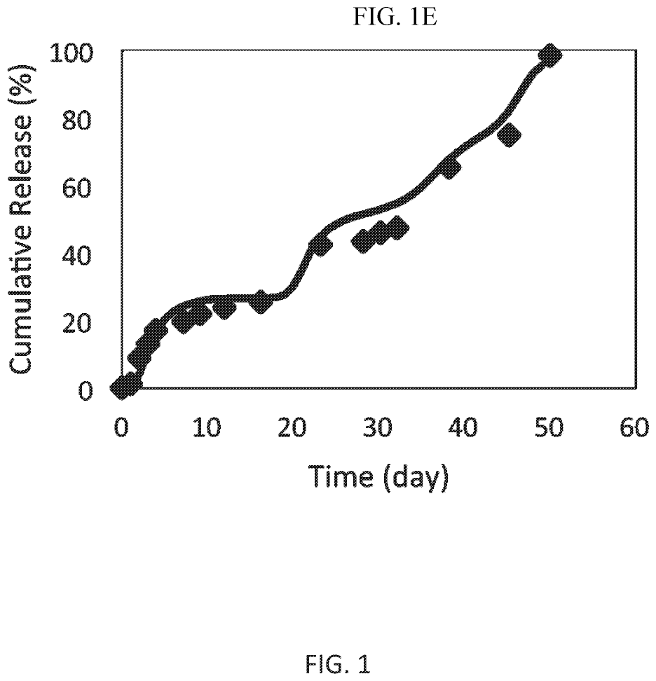

### DETAILED DESCRIPTION

Various embodiments of the invention are directed to pharmaceutical compositions for sustained release of anticancer agents and methods for using such pharmaceutical compositions. The pharmaceutical compositions may include microparticles containing an active agent that are capable of delivering therapeutic levels of the active agent to diseased tissues over a desired extended time frame, and in some embodiments, the microparticles may have different sizes and degradation profiles. Such pharmaceutical compositions may allow for continuous delivery of therapeutically effective amounts of the active agent for time periods ranging from several weeks to several months.

In certain embodiments, the size and degradation profile of the microparticles of the pharmaceutical compositions may allow for both local and regional delivery of the active agent, i.e. “dual activity” pharmaceutical compositions. In some embodiments, the pharmaceutical compositions may include microparticles of similar diameters. In other embodiments, the compositions may have microparticles of different diameters. For example, the compositions may include a mixture of microparticles of size 0.5-10 micrometers, and also larger microparticles of size greater than 10 micrometers. The compositions including a mixture of microparticles can be administered to diseased tissue, such as a cancerous tumor or to a wound resulting from the removal of a tumor. Larger microparticles may remain at the site of administration and deliver therapeutic amounts of the active agent to the administration site for a particular time period such as, for example, 30 days. Throughout this time period, smaller microparticles contained within the pharmaceutical composition may be engulfed by the antigen presenting cells, such as dendritic cells, macrophages and other peripheral monocytes, and carried to regional tissues through the lymphatic system and lymph nodes, where the active agent can be delivered to diseased cells outside of the site of administration. In so doing, the pharmaceutical compositions of various embodiments may treat cancerous tissues both at the site of a tumor and metastatic cells as they invade surrounding tissues. By varying the size of the microparticles, both the local site of administration and regional tissues surrounding a tumor can be targeted by the methods described herein. The lymph nodes are usually the primary sites for metastases, and methods described herein can be used to treat or prevent cancer metastases.

The pharmaceutical compositions of some embodiments may further treat cancerous tissues by killing or reducing the activity of immune cells and inflammatory cells associated with the cancerous tissue such as, for example, tumor-associated macrophages (“TAMs”) and cancer stem cells (“CSCs”). The activity of such cells creates a microenvironment surrounding the cancerous tissue that is suitable for cancerous tissue growth. The microenvironment may become less suitable for cancer growth by reducing the activity of these cells, causing growth of the cancerous tissue to slow and, in some cases, reverse. In some embodiments, the anti-cancer agent encapsulated by the microparticles may be an anti-cancer agent that interacts with both cancer cells and inflammatory cells and/or macrophages, reducing the activity of both cell types. In other embodiments, the pharmaceutical compositions may include small microparticles that encapsulate other active agents, such as anti-inflammatory agents, immunosuppressive agents, or anti-macrophage drugs, and large microparticles encapsulating anti-cancer agents. Without wishing to be bound by theory, the dual activity of treating cancer cells and reducing activity of anti-inflammatory cells, TAMs, and CSCs may reduce the growth of cancerous tissue and reduce or eliminate metastasis of the cancerous tissue. Small microparticles may be phagocytosed by TAMs while larger particles remain trapped within the tumor in close proximity to CSCs. Both small and large microparticles sustain the release of chemotherapeutics over six or more weeks eliminating both cell types and preventing their recurrence. In some embodiments, the active agents within the microparticles initiates immunogenic-dependent cell death of either TAMs or CSCs or both. In other embodiments, the active agents within the microparticles initiates immunogenic-dependent cell death of CSCs, and non-immunogenic death (apoptosis) of TAMs. In other embodiments, the active agents are passively targeted to TAMs and CSCs.

Dual activity pharmaceutical compositions may include microparticles of various compositions. For example, in certain embodiments, an active agent may be encapsulated in microparticles made from biodegradable polymers, such as polylactides (PLA), poly glycolides (PGA), and poly(lactide-co-glycolide) (PLGA) polymers. PLA/PGA/PLGA degrade in the body by simple hydrolysis of the ester backbone to non-harmful and non-toxic compounds. The in vivo degradation products are either excreted by the kidneys or eliminated as carbon dioxide and water through well-known biochemical pathways. Typically, the active agent can be entrapped in solid microparticles in which release of the agent is achieved by bioerosion of the microparticles.

The dual activity pharmaceutical compositions of various embodiments may contain a ratio of small microparticles (0.5-10 micrometers in diameter) to the large microparticles (10-100 micrometers in diameter) of about 1:1 (w/w), about 2:1 (w/w), about 3:1 (w/w), about 5:1 (w/w), about 10:1 (w/w), about 1:10 (w/w), about 1:5 (w/w), about 1:3 (w/w), about 1:2 (w/w) or any ratio encompassed by these example ratios. The ratio of small microparticles to large microparticles can be varied to effectuate treatment depending on the type of tumor to be treated or the probability of metastasis. For example, a higher ratio of large microparticles may be incorporated into a pharmaceutical composition used to treat a less aggressive form of cancer that is less likely to metastasize. In other embodiments, a large ratio of small microparticles may be incorporated into a pharmaceutical composition used to treat a more aggressive type of cancer that is more likely to metastasize. The increased percentage of smaller particles may allow for more regional delivery of active agent reducing the likelihood that the cancer spreads.

The average diameter of small microparticles in the pharmaceutical compositions described above can be about 0.5 micrometer to about 10 micrometers, about 0.5 micrometer to about 6 micrometers, about 0.5 micrometer to about 4 micrometers, about 0.5 micrometer to about 2 micrometers, or about 0.5 micrometer to about 1 micrometer or any range or individual diameter encompassed by these example ranges. Specific examples include about 0.5 micrometer, 1.5 micrometers, 2.5 micrometers, 3.5 micrometers, 5 micrometers, 7 micrometers, 10 micrometers, and average particle diameter there between.

The average diameter of large microparticles in the pharmaceutical compositions described above can be greater than about 10 micrometers. In some embodiments, the diameter can be from about 10 micrometers to about 100 micrometers, about 10 micrometers to about 50 micrometers, about 10 micrometers to about 25 micrometers, about 10 micrometers to about 20 micrometers, or any range or individual diameter encompassed by these example ranges. Specific examples include about 10 micrometers, 15 micrometers, 25 micrometers, 35 micrometers, 50 micrometers, 100 micrometers, and average particle diameter there between.

The term “monomodal particle size distribution” as used herein refers to a collection of particles (e.g., small microparticle and large microparticles) which have a single clearly discernable maxima on a particle size distribution curve (weight percent or population on the ordinate or Y-axis, and particle size/diameter on the abscissa or X-axis). A bimodal particle size distribution refers to a collection of particles having two clearly discernable maxima on a particle size distribution curve, and in particular embodiments, the bimodal particle distribution may have maxima at about 0.5 to about 10 micrometers and about 10 to about 100 micrometers. In some embodiments, the composition has a monomodal particle size distribution. In other embodiments, the composition has a bimodal particle size distribution, with the small microparticles forming the major mode, and the large microparticles forming the minor mode. In other embodiments, the large microparticles may form the major mode and the small microparticles may form the minor mode. In other embodiments, the composition may have multimodal particle size distribution.

In some embodiments, the composition has a monodisperse particle size distribution. In other embodiments, the composition has polydisperse particle size distribution, with a mean particle size diameter of 20 micrometers, and a standard deviation of 20 micrometers (measure of breadth of the distribution curve). In other embodiments, the composition has polydisperse particle size distribution, with a mean particle size diameter of 20 micrometers, and a standard deviation of 40 micrometers. In some embodiments, the composition has polydisperse particle size distribution, with a mean particle size diameter from about 20 micrometers to about 40 micrometers, and a standard deviation of 20 micrometers. In some embodiments, the composition has polydisperse particle size distribution, with a mean particle size diameter from about 20 micrometers to about 40 micrometers, and a standard deviation of about 20 micrometers to about 40 micrometers.

The rate of degradation may vary among various embodiments and within populations of microparticles in the pharmaceutical compositions described above. The molecular weight of the PLA/PGA/PLGA polymer units that make up the microparticles can affect the rate of degradation of the microparticle and subsequent release of the drug. For example, microparticles composed of polymer units having low molecular weights generally degrade faster and release the drug at an earlier period when compared to microparticles composed of polymer with high molecular weight polymer units. In various embodiments, the pharmaceutical compositions may include both small diameter and large diameter microparticles made from low molecular weight polymer units and both small diameter and large diameter made from high molecular weight polymers. By incorporating small diameter and large diameter microparticles having different compositions the composition of the microparticles in the pharmaceutical compositions of certain embodiments may demonstrate continuous release of an active agent, thus producing a substantially constant concentration of active agent at the site of administration and in surrounding tissues for extended time periods. This reduces the need for repeated administration.

In various embodiments, the microparticles may be composed of polymer units having molecular weights of from about 5 kDa to about 150 kDa, about 5 kDa to about 125 kDa, about 5 kDa to about 100 kDa, about 5 kDa to about 75 kDa, about 5 kDa to about 50 kDa, about 5 kDa to about 25 kDa, or about 5 kDa to about 15 kDa. Specific examples include about 5 kDa, about 17 kDa, about 25 kDa, about 33 kDa, about 42 kDa, about 52 kDa, about 53 kDa, about 63 kDa, about 70 kDa, about 124 kDa, about 150 kDa, and ranges between any of these example values. In some embodiments, the pharmaceutical compositions described above may be composed of particles substantially formed from high molecular weight polymer units having an average molecular weight per polymer unit of about 50 kDa to about 150 kDa, and particles substantially formed from low molecular weight polymer units having an average molecular weight per polymer unit of about 5 kDa to about 50 kDa. The ratio of microparticles composed of high molecular weight polymer units to microparticles composed of low molecular weight polymer units in such compositions may vary and can be about 1:1 (w/w), about 2:1 (w/w), about 3:1 (w/w), about 5:1 (w/w), about 10:1 (w/w), about 1:10 (w/w), about 1:5 (w/w), about 1:3 (w/w), about 1:2 (w/w) or any ratio encompassed by these example ratios. Modifying the ratio of microparticles composed of high molecular weight polymer units to microparticles composed of low molecular weight polymer units may modify the release profile of the active agent allowing for timed release of the active agent at various time points following administration.

The release profile of the active agent may be further modified based on the type of polymer units that make up the microparticles or the combination of polymer units incorporated in the microparticles. The microparticles of the current disclosure are not limited to poly-lactides (PLA) and poly-glycolides (PGA). They may also include derivatives of PLA or PGA, such as poly butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG).

In some embodiments, the ratio of poly-lactide to poly-glycolide in the microparticles is about 30:70 to about 99:1 by weight, about 30:70 to about 90:10 by weight, about 30:70 to about 70:30 by weight, about 30:70 to about 60:40 by weight, about 30:70 to about 50:50 by weight, or about 30:70 to about 40:60 by weight. Specific example ratios of poly-lactide to poly-glycolide include about 30:70 by weight, about 40:60 by weight, 50:50 by weight, about 60:40 by weight, about 70:30 by weight, about 75:25 by weight, about 80:20 by weight, about 99:1 by weight, and ranges between any two of these values. Microparticles having a higher concentration of lactide units degrade more slowly allowing for delayed release of the active agent.

The molecular weight and composition of the microparticles may allow the microparticles to be impermeable, and in certain embodiments, substantially all of the small diameter microparticles may be impermeable. Impermeability allows the active agent to be released following transport away from the site of administration without leaking and potentially killing cells that transport the microparticles.

The microparticles (both small and large microparticles) may be present in about 2 weight % to about 60 weight %, about 2 weight % to about 50 weight %, about 2 weight % to about 40 weight %, about 10 weight % to about 30 weight %, or about 10 weight % to about 20 weight % of the total pharmaceutical composition. Specific examples include about 2 weight %, about 15 weight %, about 25 weight %, about 45 weight %, about 60 weight % of the total pharmaceutical composition, and ranges between any two of these values.

The pharmaceutical compositions disclosed herein provide for a burst-free, sustained release of one or more anticancer agents. “Burst-free” refers to the process wherein the anticancer agent is not released from the microparticles in large quantities in the initial stage, but are released gradually over a long period of time. For example, in some embodiments the anticancer agents released from the microparticles in the initial stage is less than 10% of the total concentration of agent present within the microparticle. In some embodiments, the anticancer agents released from the microparticles in the initial stage is less than 20%-50% of the total concentration of agent present within the microparticle.

Sustained (or controlled) release refers to the gradual release of a bioactive agent from the microparticles/composition over a period of time. While there may be an initial burst phase, in some embodiments, it is preferred that the release display relatively linear kinetics, thereby providing a constant supply of the anticancer agent over the release period. The release period may vary from several hours to several months, depending upon the anticancer agent and its intended use. It is desirable that the cumulative release of the anticancer agent from the composition over the treatment period be relatively high to avoid the need for excessive loading of the microparticles and consequent waste of unreleased anticancer agent. The duration of the release period may be controlled by, inter alia, the mass and geometry of the microparticle, the concentration of the agent, the locus of administration, the molecular weight and molar composition of the microparticles, and, as demonstrated herein, the addition of release profile modifying agents.

The sustained release of the anticancer agent at a near constant rate may occur after sufficient time has elapsed after administration. For example, the release of the anticancer agent may start after about 24 hours or more after administration, about 48 hours or more after administration, about 72 hours or more after administration, about 1 week or more after administration, about 2 weeks or more after administration, about 5 weeks or more after administration, 6 weeks or more after administration, 7 weeks or more after administration, 8 weeks or more after administration, 9 weeks or more after administration, or about 10 weeks or more after administration.

Once the anticancer agent begins to release from the microparticles, the release process may continue for additional time period (sustained release period). For example, sustained release period may be for about 1 week to about 15 weeks, about 1 week to about 14 weeks, about 1 week to about 12 weeks, about 1 week to about 10 weeks, about 1 week to about 8 weeks, about 1 week to about 7 weeks, about 1 week to about 6 weeks, about 1 week to about 5 weeks, about 1 week to about 4 weeks, about 1 week to about 3 weeks, or about 1 week to about 2 weeks. During this sustained release period, drug delivery proceeds at a near constant rate. For example, for a period of about 1 week to 7 weeks, about 2% of drug may be released over a period of 24 hrs. In other embodiments, for a period of about 1 week to 6 weeks, about 3% of drug will be released over 24 hrs. In additional embodiments, for a period of about 1 week to 4 weeks, about 4% of drug will be released over 24 hrs. In further embodiments, for a period of about 1 week to 2 weeks, about 10% of drug will be released over 24 hrs.

To be more clear about the kinetics of the delayed release of the compositions, for example, the composition may release the anticancer agent starting at day 8 after the administration, and continue until day 21 after administration. In another embodiment, the composition may release the anticancer agent starting at day 8 after the administration, and continue until day 28. In another embodiment, the composition may release the anticancer agent starting at day 8 after the administration, and continue until day 50. In another embodiment, the composition may release the anticancer agent starting at day 8 after the administration, and continue until day 60. In another embodiment, the composition may release the anticancer agent starting at day 8 after the administration, and continue until day 90.

In another embodiment, the composition may release the anticancer agent starting at day 15 after the administration, and continue until day 21. In another embodiment, the composition may release the anticancer agent starting at day 15 after the administration, and continue until day 30. In another embodiment, the composition may release the anticancer agent starting at day 15 after the administration, and continue until day 50. In another embodiment, the composition may release the anticancer agent starting at day 15 after the administration, and continue until day 60. In another embodiment, the composition may release the anticancer agent starting at day 15 after the administration, and continue until day 90.

In another embodiment, the composition may release the anticancer agent starting at day 21 after the administration, and continue until day 30. In another embodiment, the composition may release the anticancer agent starting at day 21 after the administration, and continue until day 50. In another embodiment, the composition may release the anticancer agent starting at day 21 after the administration, and continue until day 60. In another embodiment, the composition may release the anticancer agent starting at day 15 after the administration, and continue until day 90.

In another embodiment, the composition may release the anticancer agent starting at day 30 after the administration, and continue until day 40. In another embodiment, the composition may release the anticancer agent starting at day 30 after the administration, and continue until day 50. In another embodiment, the composition may release the anticancer agent starting at day 30 after the administration, and continue until day 60. In another embodiment, the composition may release the anticancer agent starting at day 30 after the administration, and continue until day 90. In another embodiment, the composition may release the anticancer agent starting at day 30 after the administration, and continue until day 120.

In another embodiment, the composition may release the anticancer agent starting at day 50 after the administration, and continue until day 60. In another embodiment, the composition may release the anticancer agent starting at day 50 after the administration, and continue until day 75. In another embodiment, the composition may release the anticancer agent starting at day 50 after the administration, and continue until day 90. In another embodiment, the composition may release the anticancer agent starting at day 50 after the administration, and continue until day 120. In another embodiment, the composition may release the anticancer agent starting at day 50 after the administration, and continue until day 150.

In some embodiments, the drug delivery (drug release from microparticles) may not be at a constant rate. For example, the drug release may occur at a faster rate initially, for a period of 1-2 weeks, and later slow down. In other embodiments, the drug release may occur at a slow rate initially for 1-5 days, and later occur at a faster rate. In some embodiments, the drug release may occur in a pulse manner, with periodic intervals of no-release. Such modifications can be performed by one skilled in the art by varying the concentrations or chemistries of poly-lactides and poly-glycolides in the polymer of the microparticle.

In some embodiments, the drug release kinetics of the smaller and larger microparticles in dual activity pharmaceutical compositions may be different. For example, the smaller microparticle (0.5-5 micrometers) may release the anticancer agent starting at day 8 after the administration, and the larger microparticle (10-100 micrometers) may release the anticancer agent at day 20 after administration. In another embodiment, the smaller microparticle (0.5-5 micrometers) may release the anticancer agent starting at day 15 after the administration, and the larger microparticle (10-100 micrometers) may release the anticancer agent at day 20 after administration. In another embodiment, the smaller microparticle (0.5-5 micrometers) may release the anticancer agent starting at day 25 after the administration, and the larger microparticle (10-100 micrometers) may release the anticancer agent at day 20 after administration. In another embodiment, the smaller microparticle (0.5-5 micrometers) may release the anticancer agent starting at day 35 after the administration, and the larger microparticle (10-100 micrometers) may release the anticancer agent at day 20 after administration. In another embodiment, the smaller microparticle (0.5-5 micrometers) may release the anticancer agent starting at day 15 after the administration, and the larger microparticle (10-100 micrometers) may release the anticancer agent at day 30 after administration. In another embodiment, the smaller microparticle (0.5-5 micrometers) may release the anticancer agent starting at day 50 after the administration, and the larger microparticle (10-100 micrometers) may release the anticancer agent at day 70 after administration.

In some embodiments, the dual activity pharmaceutical compositions may contain a single active agent or a mixture of one or more active agents. For example, in some embodiments, small microparticles and large microparticles may encapsulate the same active agent. In other embodiments, small microparticles may encapsulate a first anticancer agent, and large microparticles may encapsulate a second anticancer agent. In still other embodiments, both the small microparticles and large microparticles may encapsulate a first and a second anticancer agent. This allows targeting both the local site of administration and regional sites with different therapeutic regimen and dose. In still other embodiments, the small microparticles may encapsulate an anti-inflammatory, immunosuppressive, or anti-macrophage agent and the large microparticles may encapsulate an anticancer agent.

The microparticles may encapsulate one or more active agents, and in certain embodiments, the active agent may be an anti-cancer agent. Non-limiting examples of anticancer agents that be used in the compositions described herein are tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, metronicdazole, camptothecin, vincristine, taxol, taxotere, etoposide, teniposide, amsacrine, Irinotecan, topotecan, epothilones, gefitinib, erlotinib, angiogenesis inhibitors, EGF inhibitors, VEGF inhibitors, CDK inhibitors, cytokines, Her1 inhibitors, Her2 inhibitors, and monoclonal antibodies.

Other anticancer agents that may be encapsulated without limitation are 2,2′,2″trichlorotriethylamine, 6-azauridine, 6-diazo-5-oxo-L-norleucine, mercaptopurine, aceglarone, aclacinomycinsa actinomycin, altretamine, aminoglutethimide, amsacrine, anastrozole, ancitabine, angiogenin antisense oligonucleotide, anthramycin, azacitidine, azaserine, aziridine, batimastar, bcl-2 antisense oligonucleotide, benzodepa, bicalutamide, bisantrene, bleomycin, buserelin, busulfan, cactinomycin, calusterone, carboplatin, carboquone, carmofur, carmustine, carubicin, carzinophilin, chlorambucil, chloraphazine, chlormadinone acetate, chlorozotocin, chromomycins, cisplatin, cladribine, cyclophosphamide, cytarabine, dacarbazine, dactinomycin, daunorubicin, defosfamide, demecolcine, denopterin, diaziquone, docetaxel, doxifluridine, doxorubicin, droloxifene, dromo-stanolone, edatrexate, eflornithine, elliptinium acetate, emitefur, enocitabune, epirubicin, epitiostanol, estramustine, etoglucid, etoposide, fadrozole, fenretinide, floxuridine, fludarabine, fluorouracil, flutamide, folinic acid, formestane, fosfestrol, fotemustine, gallium nitrate, gemcitabine, goserelin, hexestrol, hydroxyurea, idarubicin, ifosfamide, improsulfan, interferonalpha, interferonbeta, interferon-gamma, interleukin-2, L-asparaginase, lentinan, letrozole, leuprolide, lomustine, lonidamine, mannomustine, mechlorethamine, mechlorethamine oxide hydrochloride, medroxyprogesterone, megestrol acetate, melengestrol, melphalan, menogaril, mepitiostane, methotrexate, meturedepa, miboplatin, miltefosine, mitobronitol, mitoguazone, mitolactol, mitomycins, mitotane, mitoxantrone, mopidamol, mycophenolic acid, nilutamide, nimustine, nitracine, nogalamycin, novembichin, olivomycins, oxaliplatin, paclitaxel, pentostain, peplomycin, perfosfamide, phenamet, phenesterine, pipobroman, piposulfan, pirarubicin, piritrexim, plicamycin, podophyllinic acid 2-ethyl-hydrazide, polyestradiol phosphate, porfimer sodium, porfiromycin, prednimustine, procabazine, propagermanium, PSK, pteropterin, puromycin, ranimustine, razoxane, roquinimex, sizofican, sobuzoxane, spirogerma-nium, streptonigrin, streptozocin, tamoxifen, tegafur, temozolomide, teniposide, tenuzonic acid, testolacone, thiamiprine, thioguanine, Tomudex, topotecan, toremifene, triaziquone, triethylenemelamine, triethylenephosphoramide, triethylenethiophosphoramide, trilostane, trimetrexate, triptorelin, trofosfamide, trontecan, tubercidin, ubenimex, uracil mustard, uredepa, urethan, vincristine, zinostatin, zorubicin, cytosine arabinoside, gemtuzumab, thioepa, cyclothosphamide, antimetabolites (e.g., methotrexate, 6-mercaptopurine, 6-thioguanine, cytarabine, 5-fluorouracil, fludarabine, gemcitabine, dacarbazine, temozoamide), hexamethylmelamine, LYSODREN, nucleoside analogues, plant alkaloids (e.g., Taxol, paclitaxel, camptothecin, topotecan, irinotecan (CAMPTOSAR,CPT-11), vinca alkyloids such as vinblastine, podophyllotoxin, epipodophyllotoxin, VP-16 (etoposide), cytochalasin B, gramicidin D, ethidium bromide, emetine, anthracyclines, liposomal doxorubicin, dihydroxyanthracindione, mithramycin, actinomycin D, aldesleukin, allutamine, biaomycin, capecitabine, carboplain, chlorabusin, cyclarabine, daclinomycin, floxuridhe, lauprolide acetate, levamisole, lomusline, mercaptopurino, mesna, mitolanc, pegaspergase, pentoslatin, picamycin, riuxlmab, campath-1, straplozocin, tretinoin, VEGF antisense oligonucleotide, vindesine, and vinorelbine. Compositions comprising one or more anticancer agents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone. In certain embodiments, the anticancer agent may have both anti-cancer properties and anti-inflammatory, immunosuppressive, or anti-macrophage properties. Such drugs include, for example, epothilones (A-F), doxorubicin, oxaliplatin, bleomycin, cyclophosphamide, bortezomib, and the like.

The microparticles can also be used encapsulate other active agents, such as poly-peptide antibiotics, antimalarials, antituberculosis drugs, anesthetics, analgesics, antiparasitic agents, antibacterial agents, antifungal agents, antiinflammatory agents, immunosuppressive agents, immunostimulatory agents, dentinal desensitizers, odor masking agents, nutritional agents, antioxidants, and insulin. In particular embodiments, the microparticles may encapsulate anti-inflammatory agents, immunosuppressive agents, or anti-macrophage agents, and in some embodiments, the small microparticles may encapsulate anti-inflammatory agents, immunosuppressive agents, anti-macrophage agents, or combinations thereof.

The amount or the concentration of the anticancer agent encapsulated by PLGA microparticle may vary, depending on the use and the site of administration. In some embodiments, 1 milligram of microparticles may encapsulate about 0.01 microgram to about 100 micrograms of one or more anticancer agents. Specific examples include about 0.1 microgram, about 0.5 microgram, about 1 microgram, about 5 micrograms, about 10 micrograms, about 20 micrograms, about 50 micrograms, about 100 micrograms, and ranges between any two of these values.

In some embodiments, the ratio of PLGA to the anticancer agent may be from about 0.001:1 weight % to about 9:1 weight %, about 0.001:1 weight % to about 5:1 weight %, or about 0.001:1 weight % to about 0.05:1 weight %. Specific examples include about 0.001:1 weight %, about 0.005:1 weight %, about 0.01:1 weight %, about 0.05:1 weight %, about 0.1:1 weight %, about 0.5:1 weight %, about 1:1 weight %, about 2:1 weight %, about 3:1 weight %, about 4:1 weight %, about 9:1 weight %, and ranges between any of the two values.

The anticancer agents disclosed herein may target one or more cancer cells, such as a colorectal cancer cell, a breast cancer cell, an ovarian cancer cell, a pancreatic cancer cell, a head and neck cancer cell, a bladder cancer cell, a liver cancer cell, a renal cancer cell, a melanoma cell, a gastrointestinal cancer cell, a prostate cancer cell, a small cell lung cancer cell, non-small cell lung cancer cell, a sarcoma cell, a glioblastoma cell, T- and B-cell lymphoma cell, a endometrial cancer cell, and a cervical cancer cell.

The microparticles and encapsulation of anticancer agents may be performed as follows. Briefly, PLGA polymers of known molecular weights are dissolved in organic solvents, such as halogenated hydrocarbons, for example, methylene chloride, chloroform, and carbon tetrachloride; aromatic hydrocarbons, such as toluene and xylene; and mixed solvents thereof. One or more anticancer agents may be dissolved in an aqueous solvent, such as polyvinyl alcohol, polyvinyl pyrrolidone, carboxymethyl cellulose, lecithin, and gelatin. The PLGA solution and the anticancer agent solution are mixed and sonicated to form a uniform distribution of the anticancer agent and the PLGA polymer. Homogenization is subsequently performed to form the polymer particle emulsion. The resulting polymer emulsion is stirred until the organic solvent is evaporated, resulting in precipitation of polymer particles that encapsulate the anticancer agent. The size of the microparticles may be controlled by varying homogenization speed during emulsification.

### Hydrogel

The sustained release pharmaceutical compositions disclosed herein may further contain a hydrogel. The hydrogel may help to hold the microparticles in the composition without clumping. The hydrogel may serve to maintain the integrity of the microparticles by buffering the pH. The hydrogel may also aid in administration of the composition. Non-limiting examples of hydrogels include methyl cellulose (MC), ethyl cellulose (EC), ethyl methyl cellulose (EMC), hydroxyethyl cellulose (HEC), hydroxylpropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), and hydroxyethylcarboxymethyl cellulose (HECMC).

Other materials that can be used to form a hydrogel include modified alginates. Alginate is a carbohydrate polymer isolated from seaweed, which can be crosslinked to form a hydrogel by exposure to a divalent cation, such as calcium. Additionally, polysaccharides which gel by exposure to monovalent cations, including bacterial polysaccharides, such as gellan gum, and plant polysaccharides, such as carrageenans, may be crosslinked to form a hydrogel, using methods known in the art. Tragacanth, pectin, guar gum, xanthan gum, and polyacrylamide may also be used as hydrogels.

In some embodiments, the sustained release compositions may have an inherent viscosity of about 300 cP or less, about 200 cP or less, about 100 cP or less or about 50 cP or less. Combinations of viscosity reducing agents may be used to achieve the desired viscosity. For example, polyethylene glycol polymers, surfactants, organic solvents, aqueous solvents and combinations thereof are suitable for use as viscosity reducing agents. The amount of viscosity reducing agent present in the sustained release composition can range from about 5 wt % to about 40 wt % of the total weight of the sustained release composition.

### Formulations

Formulations of the present sustained release pharmaceutical compositions can be in forms which include, but are not limited to, solutions, softgels, tablets, capsules, cachets, pellets, pills, powders and granules. Topical dosage forms which include, but are not limited to, solutions, powders, fluid emulsions, fluid suspensions, semi-solids, ointments, pastes, creams, gels and jellies, and foams. Parenteral dosage forms which include, but are not limited to, solutions, suspensions, emulsions, and dry powder. Formulations can also be in the form of films, pads, wafers, injectables, hydrogels, and the like.

The pharmaceutical compositions of the invention are typically used in the form of a drug reservoir such as injectable microparticles, passive transdermal/transmucosal drug delivery or electrotransport drug delivery systems. It will be appreciated by those skilled in the art that the inventive formulations described herein can be combined with suitable carriers to prepare alternative drug dosage forms (e.g., oral capsule, topical ointment, rectal and/or vaginal suppositories, buccal patches, or an aerosol spray).

It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

Pharmaceutical compositions disclosed herein can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols. In some embodiments, the pharmaceutical excipient may include, without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like.

In some embodiments, the pharmaceutical composition may include one or more disintegrant component, such as croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floe, carboxymethylcellulose, hydroxypropylcellulose, calcium silicate, a metal carbonate, sodium bicarbonate, calcium citrate, or calcium phosphate.

In some embodiments, the pharmaceutical composition may include one or more diluent component, such as mannitol, lactose, sucrose, maltodextrin, sorbitol, xylitol, powdered cellulose, microcrystalline cellulose, carboxymethyl-cellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the pharmaceutical composition may include one or more optional lubricant component, such as stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### Administration

Disclosed herein are methods to treat or prevent cancer in a subject. In one embodiment, a method of treating cancer in a subject involves administering a therapeutically effective amount of a pharmaceutical composition disclosed herein, wherein the composition provides a burst-free, sustained release of one or more anticancer agents beginning at 2 weeks after administration, and wherein the composition comprises a plurality of poly(lactic-co-glycolic) acid (PLGA) microparticles encapsulating the one or more anticancer agents.

Non-limiting examples of anticancer agents include tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, metronicdazole, camptothecin, vincristine, taxol, taxotere, etoposide, teniposide, amsacrine, Irinotecan, topotecan, epothilones, gefitinib, erlotinib, angiogenesis inhibitors, EGF inhibitors, VEGF inhibitors, CDK inhibitors, cytokines, Her1 inhibitors, Her2 inhibitors, and monoclonal antibodies.

In another embodiment, a method of treating a subject with cancer includes administering a therapeutically effective amount of a pharmaceutical composition comprising microparticles having a bimodal particle size distribution, wherein the microparticles encapsulate one or more anticancer agents.

In an additional embodiment, a method of treating a subject with cancer includes administering a therapeutically effective amount of a pharmaceutical composition comprising microparticles having multimodal particle size distribution, wherein the microparticles encapsulate one or more anticancer agents.

The term “subject” includes animals which can be treated using the methods of the invention. Examples of animals include mammals, such as mice, rabbits, rats, horses, goats, dogs, cats, pigs, cattle, sheep, and primates (e.g. chimpanzees, gorillas, and, preferably, humans). In a further embodiment, the patient is a cancer patient, e.g., a human suffering from cancer, a tumor or tumors.

The subject treated for cancer may be suffering from, but not limited to, colorectal cancer, breast cancer, ovarian cancer, pancreatic cancer, head and neck cancer, bladder cancer, liver cancer, renal cancer, melanoma, gastrointestinal cancer, prostate cancer, small cell lung cancer, non-small cell lung cancer, sarcoma, glioblastoma, T- and B-cell lymphoma, endometrial cancer, and cervical cancer.

In some embodiments, a method of treating or preventing cancer in a subject involves administering a dose of first anticancer agent to the patient and allowing a time ranging from 1 to 7 days to elapse before administering a sustained release composition comprising a second anticancer agent. The sustained release composition comprises a plurality of poly(lactic-co-glycolic) acid (PLGA) microparticles encapsulating the second anticancer agent. In some embodiments, the first and the second anticancer agent are same. In some embodiments, the first and the second anticancer agent are different.

In some embodiments, the method includes administering the sustained release composition as an adjuvant therapy. In some embodiments, the method includes administering the sustained release composition as a neo-adjuvant therapy. In some embodiments, the sustained release composition can be administered with other treatments, such as radiation therapy, chemotherapy, targeted therapy, gene therapy, or hormone therapy.

In some embodiments, the sustained release compositions can be used in combination with other anticancer agents that are administered systemically. In another embodiment, the sustained release compositions may be administered in combination with one or more cytokines which include, without limitation, a lymphokine, tumor necrosis factors, tumor necrosis factor-like cytokine, lymphotoxin, interferon, macrophage inflammatory protein, granulocyte monocyte colony stimulating factor, interleukins including, without limitation, interleukin-1, interleukin-2, interleukin-6, interleukin-12, interleukin-15, and interleukin-18.

Administration can be systemic, parenteral, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. In other embodiments, administration can be at the site of tumor resection. Thus, modes of administration of the composition of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compounds to be administered is that amount which is therapeutically effective. The dosage to be administered will depend on the characteristics of the subject being treated, e.g., the particular animal or human being treated, age, weight, health, types of concurrent treatment, if any, and frequency of treatments, and can be easily determined by one of skill in the art (e.g., by the clinician).

The dimensions of the sustained release compositions may be commensurate with the size and shape of the region selected as the site of administration and will not migrate from the insertion site following implantation, injection or other means of depot administration. The sustained release compositions may be rigid, or somewhat flexible so as to facilitate both insertion of the implant at the target site and accommodation of the implant. The sustained release compositions may be particles, sheets, patches, plaques, fibers, microcapsules and the like and may be of any size or shape compatible with the selected site of insertion.

In one embodiment, the sustained release composition may be administered by a catheter. In another embodiment, the sustained release composition may be administered by a syringe. The sustained release composition is formulated so that the composition can be readily implanted (e.g., by injection) into the desired location to form a mass that can remain in place for the period suitable for controlled release of the anticancer agent and for any additional benefit of mechanical support if applicable. The mechanical and rheological properties suitable for injectable depot compositions are known in the art. Typically, the polymer of the depot vehicle with particulates are present in an appropriate amount of solvent such that the depot composition can be so implanted.

An alternative embodiment of the invention provides for a rod depot implant. Other embodiments include a drug depot implant comprising a hollow depot, the hollow depot comprising a therapeutic agent that provides a concentration gradient for targeted delivery of the agent to the synovial joint, the disc space, the spinal canal, or the soft tissue surrounding the spinal canal of a subject.

For oral administration, the pharmaceutical composition can be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

For oral administration, the hydrogel formulation is preferably encapsulated by a retardant coating, e.g., a bioerodible polymer. Upon dissolution or erosion of the encapsulating material, the hydrogel core becomes exposed and the drug contained within the gel can be released for enteric adsorption. Bioerodible coating materials may be selected from a variety of natural and synthetic polymers, depending on the agent to be coated and the desired release characteristics. Exemplary coating materials include gelatins, carnauba wax, shellacs, ethylcellulose, cellulose acetate phthalate or cellulose acetate butyrate. Release of the agent is controlled by adjusting the thickness and dissolution rate of the polymeric coat.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions of the present invention can also be formulated in rectal compositions such as suppositories or retention enemas, e.g., containing conventional suppository bases such as cocoa butter or other glycerides.

In addition to the formulations described previously, the compositions of the present invention can also be formulated as a depot preparation. Such long acting formulations can be administered by implantation (for example subcutaneously or intramuscularly) or by intramuscular injection.

In transdermal administration, the compositions of the present invention, for example, can be applied to a plaster, or can be applied by transdermal, therapeutic systems that are consequently supplied to the organism.

In various embodiments of the invention, the sustained release composition is directly administered to the area of the tumor(s) by, for example, local infusion during surgery, topical application (e.g., in conjunction with a wound dressing after surgery), injection, means of a catheter, means of a suppository, or means of an implant. An implant can be of a porous, non-porous, or gelatinous material, including membranes, such as sialastic membranes, or fibers. Suppositories generally contain active ingredients in the range of 0.5% to 10% by weight.

In other embodiments, a controlled release system can be placed in proximity of the target tumor. For example, a micropump may deliver controlled doses directly into the area of the tumor, thereby finely regulating the timing and concentration of the pharmaceutical composition.

### Packs and Kits

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers having therapeutically or prophylactically effective amounts of the sustained release compositions in pharmaceutically acceptable form. The sustained release compositions in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the complex, and/or a packaged alcohol pad. Instructions are optionally included for administration of sustained release compositions by a clinician or by the patient.

### Dosage

In some embodiments, about 1 microgram to 500 grams of the sustained release composition is administered. In some embodiments, about 1 microgram to 400 grams of the sustained release composition is administered. In some embodiments, about 1 microgram to 300 grams of the sustained release composition is administered. In some embodiments, about 1 microgram to 200 grams of the sustained release composition is administered.

In one embodiment, the effective dose of the anticancer agent that is released from the sustained release compositions may range from about 0.1 to 3000, 0.2 to 900, 0.3 to 800, 0.4 to 700, 0.5 to 600, 0.6 to 500, 70 to 400, 80 to 300, 90 to 200, or 100 to 150 micrograms/day. In other embodiments, the dose may range from approximately 10 to 20, 21 to 40, 41 to 80, 81 to 100, 101 to 130, 131 to 150, 151 to 200, 201 to 280, 281 to 350, 351 to 500, 501 to 1000, 1001 to 2000, or 2001 to 3000 nanograms/day. In specific embodiments, the dose may be at least approximately 20, 40, 80, 130, 200, 280, 400, 500, 750, 1000, 2000, or 3000 micrograms/dose. In specific embodiments, the dose may be at least approximately 20, 40, 80, 130, 200, 280, 400, 500, 750, 1000, 2000, or 3000 nanograms/dose.

In another embodiment, the effective dose of the anticancer agent that is released results in a plasma concentration of approximately 0.1, 1, 2.5, 5, 7.5, 10, 15, 20, 30, 40, or 50 micrograms/liter. In another embodiment, the effective dose of the anticancer agent that is released results in a plasma concentration of approximately 0.1, 1, 2.5, 5, 7.5, 10, 15, 20, 30, 40, or 50 nanograms/liter. In other embodiments, the resulting circulating concentration of the anticancer agent is approximately 0.1 to 50, 1 to 40, 2.5 to 30, 5 to 20, or 7.5 to 10 micrograms/liter. In other embodiments, the resulting circulating concentration of the anticancer agent is approximately 0.1 to 50, 1 to 40, 2.5 to 30, 5 to 20, or 7.5 to 10 nanograms/liter. In other embodiments, the resulting circulating concentration of the anticancer agent is approximately 0.1 to 1, 1.1 to 2.4, 2.5 to 5, 5.1 to 7.4, 7.5 to 10, 11 to 15, 16 to 20, 21 to 30, 31 to 40, or 41 to 50 micrograms/liter. In other embodiments, the resulting circulating concentration of the anticancer agent is approximately 0.1 to 1, 1.1 to 2.4, 2.5 to 5, 5.1 to 7.4, 7.5 to 10, 11 to 15, 16 to 20, 21 to 30, 31 to 40, or 41 to 50 nanograms/liter.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples. The following examples are for illustrative purposes only and are not to be construed as limiting the invention in any manner.

### Example 1

### Preparation of Epothilone D Loaded Microparticles (QR206)

Epothilone D (EpoD) and PLGA were dissolved in dichloromethane and mixed with 2% polyvinyl alcohol (PVA). This mixture was sonicated and then homogenized (<1.5 min total) to form a micro emulsion. The dichloromethane was allowed to evaporate over 3 hours, precipitating microparticles. These particles were collected by centrifugation and were washed ×4 in deionized water to remove PVA before being aliquoted for storage at −20° C.

To determine the drug loading in the microparticles, a known mass of microparticles was dissolved in 1 mL of methanol with 0.43% orthophosphoric acid. A 700 μL aliquot was then diluted with HPLC grade water to form a 70/30 v/v with 0.3% orthophosphoric acid. EpoD concentration in the solution was measured by HPLC. Average loading of EpoD per mass of microparticle was measured as 1.7±0.6 μg/mg across three different batches of microparticles.

### Example 2

### Xenograft Studies

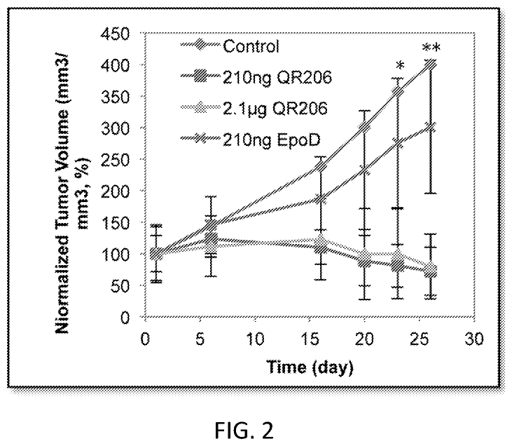

Athymic mice were injected subcutaneously with head and neck squamous cell carcinoma cells to form tumors. Mice were divided into groups and treated with a sustained release composition according to the present invention, QR206, at a high dose (2.1 micrograms) a low dose (210 nanograms), or with a low dose of unformulated drug (epothilone D, 210 nanograms). Tumor volume was monitored for 4 weeks. Both high and low dose QR206 achieve statistically significant reductions in tumor volume relative to the unformulated drug control (T-test, *p<0.04, ** p<0.02) (FIG. 2).

1-29. (canceled)

30. A pharmaceutical composition comprising a population of small microparticles and a population of large microparticles,
wherein the small microparticles and the large microparticles are comprised of poly(lactic-co-glycolic) acid (PLGA) polymer units;
wherein the large microparticles encapsulate an anticancer agent and have a mean particle diameter of about 10 to about 100 micrometers, and wherein the PLGA polymer units of the large microparticles have a molecular weight of about 17 kDa to about 25 kDa;
wherein the small microparticles encapsulate an immunosuppressive agent or an anti-inflammatory agent and have a mean particle diameter of about 0.5 to about 10 micrometers, and wherein the PLGA polymer units of the small microparticles have a molecular weight of about 5 kDa to about 15 kDa; and
wherein the small microparticles and the large microparticles comprise PLGA at a ratio of poly-lactide to poly-glycolide of 30:70 to 70:30 by weight.

31. The pharmaceutical composition of claim 30, wherein the anticancer agent is doxorubicin or cyclophosphamide.

32. The pharmaceutical composition of claim 31, wherein the small microparticles encapsulate an immunosuppressive agent or an anti-inflammatory agent.

33-34. (canceled)

35. The pharmaceutical composition of claim 30, wherein the ratio of poly-lactide to poly-glycolide is 50:50 by weight.

36. A method of reducing tumor volume in a subject, the method comprising administering to the subject a therapeutically effective amount of the pharmaceutical composition of claim 30, wherein the composition is administered at the site of the tumor.

37-39. (canceled)

40. A method of reducing or eliminating metastasis of cancer in a subject, the method comprising administering to the subject a therapeutically effective amount of the pharmaceutical composition of claim 30, wherein administration is at the site of the cancer or at the site of a tumor resection.

41-43. (canceled)

44. A pharmaceutical composition comprising a population of small microparticles and a population of large microparticles,
wherein the small microparticles and the large microparticles are comprised of polymer units selected from the group consisting of polylactic acid (PLA), polyglycolic acid (PGA), poly(lactic-co-glycolic) acid (PLGA), polybutylene succinate (PBS), polyhydroxyalkanoate (PHA), polycaprolactone acid lactone (PCL), polyhydroxybutyrate (PHB), glycolic amyl acid (PHV), PHB and PHV copolymer (PHBV), and PLA-polyethylene glycol (PEG) copolymer (PLEG);
wherein the large microparticles encapsulate an anticancer agent and have a mean particle diameter of about 10 to about 100 micrometers;
wherein the small microparticles encapsulate an immunosuppressive agent or an anti-inflammatory agent and have a mean particle diameter of about 0.5 to about 10 micrometers; and
wherein the composition provides sustained release of the encapsulated agents for about 1 week to about 3 weeks.

45. The pharmaceutical composition of claim 44, wherein the anticancer agent is doxorubicin or cyclophosphamide.

46. The pharmaceutical composition of claim 45, wherein the small microparticles encapsulate an immunosuppressive agent or an anti-inflammatory agent.

47-48. (canceled)

49. The pharmaceutical composition of claim 44, wherein the small microparticles and the large microparticles are comprised of PLGA polymer units.

50. The pharmaceutical composition of claim 49, wherein the PLGA polymer units of the small microparticles have a molecular weight of about 5 kDa to about 15 kDa, and wherein the PLGA polymer units of the large microparticles have a molecular weight of about 17 kDa to about 25 kDa.

51. The pharmaceutical composition of claim 49, wherein the ratio of poly-lactide to poly-glycolide in the PLGA polymer units is 50:50 by weight.

52. A method of reducing tumor volume in a subject, the method comprising administering to the subject a therapeutically effective amount of the pharmaceutical composition of claim 44, wherein the composition is administered at the site of the tumor.

53-54. (canceled)

55. A method of reducing or eliminating metastasis of cancer in a subject, the method comprising administering to the subject a therapeutically effective amount of the pharmaceutical composition of claim 44, wherein administration is at the site of the cancer or at the site of a tumor resection.

56-57. (canceled)

58. A pharmaceutical composition comprising microparticles having a polydisperse particle size distribution,
wherein the microparticles have a mean particle size diameter from about 20 micrometers to about 40 micrometers with a standard deviation of about 20 micrometers;
wherein the microparticles are comprised of poly(lactic-co-glycolic) acid (PLGA) having a ratio of poly-lactide to poly-glycolide of 30:70 to 70:30 by weight;
wherein a first population of the microparticles is comprised of PLGA polymer units having a molecular weight of about 17 kDa to about 25 kDa;
wherein a second population of the microparticles is comprised of PLGA polymer units having a molecular weight of about 42 kDa to about 63 kDa; and
wherein the microparticles encapsulate an anticancer agent.

59. The pharmaceutical composition of claim 58, wherein the anticancer agent is an epothilone.

60. (canceled)

61. The pharmaceutical composition of claim 58, wherein the ratio of poly-lactide to poly-glycolide is 50:50 by weight.

62. A method of reducing tumor volume in a subject, the method comprising administering to the subject a therapeutically effective amount of the pharmaceutical composition of claim 58, wherein administration is at the site of the tumor.

63-65. (canceled)

66. A pharmaceutical composition comprising microparticles having a polydisperse particle size distribution,
wherein the microparticles have a mean particle size diameter from about 20 micrometers to about 40 micrometers with a standard deviation of about 20 micrometers;
wherein the microparticles are comprised of are comprised of polymer units selected from the group consisting of polylactic acid (PLA), polyglycolic acid (PGA), poly(lactic-co-glycolic) acid (PLGA), polybutylene succinate (PBS), polyhydroxyalkanoate (PHA), polycaprolactone acid lactone (PCL), polyhydroxybutyrate (PHB), glycolic amyl acid (PHV), PHB and PHV copolymer (PHBV), and PLA-polyethylene glycol (PEG) copolymer (PLEG);
wherein a first population of the microparticles is comprised of polymer units having a molecular weight of about 5 kDa to about 33 kDa;
wherein a second population of the microparticles is comprised of polymer units having a molecular weight of about 42 kDa to about 150 kDa; and
wherein the microparticles encapsulate an anticancer agent.
